(19) United States
(12) Patent Application Publication
(10) Pub. No.: US 2011/0202135 A1
(43) Pub. Date: Aug. 18, 2011

(54) EXPANDABLE SPINAL INTERBODY CAGE AND METHODS

(75) Inventors: Seungwon Baek, Phoenix, AZ (US); Anna Sawa, Chandler, AZ (US); Sam Safavi-Abbasi, Oklohoma City, OK (US); Phillip M. Reyes, Mesa, AZ (US); Nicholas Theodore, Paradise Valley, AZ (US); Neil R. Crawford, Tempe, AZ (US)

(73) Assignee: Lanx, Inc.

(21) Appl. No.: 12/933,484

(22) PCT Filed: Mar. 23, 2009
(86) PCT No.: PCT/US09/37929

§ 371 Date: Apr. 28, 2011

Related U.S. Application Data
(60) Provisional application No. 61/038,813, filed on Mar. 24, 2008.

(51) Int. Cl. A61F 2/44

(52) U.S. Cl. 623/17.16

(57) ABSTRACT

An implantable device assembly including a bone fusion cage assembly having first and second housing members and a biasing member. The first housing member defines a first contact surface. The second housing member defines a second contact surface that faces generally opposite the first contact surface. The biasing member is operable to bias the first and second housing members away from each other into an expanded position. Each of the first and second housing members may include at least one pivot portion that defines the contact surfaces. The implantable device assembly may also include a plate assembly that is mounted to the bone fusion cage assembly.

### PRIORITY CLAIM

This application claims priority to U.S. Provisional Patent application Ser. No. 61/038,813, filed Mar. 24, 2008, titled Expandable Spinal Interbody Cage and Enhancements, incorporated herein by reference.

### TECHNICAL FIELD

The present disclosure generally relates to spinal implants and associated methods and, more particularly, relates to spinal interbody cage structures and related methods.

### BACKGROUND

The vertebrae of the human spine are arranged in a column with one vertebra on top of the next. An intervertebral disc lies between adjacent vertebrae to transmit force between the adjacent vertebrae and provide a cushion between them. The discs allow the spine to flex and twist. With age, spinal discs begin to break down, or degenerate, resulting in the loss of fluid in the discs and consequently resulting in them becoming less flexible. Likewise, the discs become thinner allowing the vertebrae to move closer together. Degeneration may also result in tears or cracks in the outer layer, or annulus, of the disc. The disc may begin to bulge outwardly. In more severe cases, the inner material of the disc, or nucleus, may actually extrude out of the disc. In addition to degenerative changes in the disc, the spine may undergo changes due to trauma from automobile accidents, falls, heavy lifting, and other activities. Furthermore, in a process known as spinal stenosis, the spinal canal narrows due to excessive bone growth, thickening of tissue in the canal (such as ligament), or both. In all of these conditions, the spaces through which the spinal cord and the spinal nerve roots pass may become narrowed, leading to pressure on the nerve tissue which can cause pain, numbness, weakness, or even paralysis in various parts of the body. Finally, the facet joints between adjacent vertebrae may degenerate and cause localized and/or radiating pain. All of the above conditions are collectively referred to herein as spine disease.

Conventionally, surgeons treat spine disease by attempting to restore the normal spacing between adjacent vertebrae. This may be sufficient to relieve pressure from affected nerve tissue. However, it is often necessary to also surgically remove disc material, bone, or other tissues that impinge on the nerve tissue and/or to debride the facet joints. Most often, the restoration of vertebral spacing is accomplished by inserting a rigid spacer made of bone, or biocompatible metal or plastic into the disc space between the adjacent vertebrae and allowing the vertebrae to grow together, or fuse, into a single piece of bone. The vertebrae are typically stabilized during this fusion process with the use of bone plates, spacers, grafts, and/or pedicle screws fastened to the adjacent vertebrae.

Immobilizing the superior and inferior vertebrae with a bone graft in the intervertebral disc space prompts fusion of the superior and inferior vertebrae into one solid bone. Proper positioning and immobilization of the bone graft in the intervertebral disc space can lead to improved fusion of the vertebrae bone. In some treatments, the bone graft is constructed as a cage-like device. The cage is apertured, and includes a hollow interior chamber. Following implantation, bone from each of the adjacent vertebrae grow through the apertures to fuse with the bone of the other vertebrae above and below the cage, thus stabilizing the area. Opportunities for advancement in this technical area are available.

### DISCLOSURE OF INVENTION

One aspect of the present disclosure relates to a bone fusion cage assembly that includes first and second housing members and a biasing member. The first housing member includes a first contact surface, a first hollow cylindrical shaped body, and a first pivot portion. The first hollow cylindrical shaped body has a first closed end. The first pivot portion defines an angular orientation of the first contact surface relative to the first hollow cylindrical shaped body. The second housing member includes a second contact surface, wherein the second housing member is slidably coupled to the first housing member and the second contact surface faces opposite the first contact surface. The biasing member is positioned within at least a portion of the first hollow cylindrical shaped body and is operable to bias the first and second housing members away from each other into an expanded state.

Another aspect of the present disclosure is directed to an implantable device assembly that includes a bone fusion cage assembly and an insertion tool. The bone fusion cage assembly includes first and second housing members and a biasing member. The first housing member defines a first contact surface. The second housing member defines a second contact surface, wherein the second contact surface faces generally opposite the first contact surface. The biasing member is configured to apply a biasing force to the first and second housing members. The insertion tool includes an attachment member, a release member, and an actuator. The attachment member is configured to releasably mount the insertion tool to the bone fusion cage assembly. The actuator is operable to move the release member from a first position in which the first and second housing members are retained in a compressed state relative to each other, to a second position in which the first and second housing members are movable away from each other by application of the biasing force.

A further aspect of the present disclosure relates to a method of operating a bone fusion cage assembly. The method includes providing a bone fusion cage assembly having first and second housing members and a biasing member operable between the first and second housing members. Each of first and second housing members may include a contact surface and a base portion. At least one of the first and second housing members includes a pivotal connection of the contact surface to the base portion. The method also includes moving the first and second housing members in a direction toward each other to move the biasing member into an unexpanded state, retaining the first and second housing members together with the biasing member in the unexpanded state, and permitting the biasing member to move from the unexpanded state to an expanded state to move the first and second housing members in a direction away from each other. The method may further include contacting the contact surfaces of the first and second housing member against opposing body surfaces, wherein the pivotal connection of the contact surfaces providing self-alignment of the contact surfaces with the opposing tissue surfaces.

The foregoing and other features, utilities and advantages of the invention, will be apparent from the following more particular description of a preferred embodiment of the invention as illustrated in the accompanying drawings.

### BRIEF DESCRIPTION OF THE DRAWINGS

The accompanying drawings, which are incorporated in and constitute a part of this specification, illustrate embodiments of the present invention, and together with the description, serve to explain the principles thereof. Like items in the drawings are referred to using the same numerical reference.

FIG. 1 is a perspective view of an example of an implantable device assembly according to the present disclosure;

FIG. 2 is a top view of a portion of the implantable device assembly of FIG. 1;

FIG. 2A is a cross-sectional view of the bone fusion cage assembly of FIG. 1 in a compressed state;

FIG. 3 is a top view of a portion of an insertion tool of the implantable device assembly of FIG. 1;

FIG. 4 is an front perspective view of the implantable device assembly of FIG. 1;

FIG. 4A is a cross-sectional view of the bone fusion cage assembly of FIG. 4 in a partially expanded state;

FIG. 5 is an front perspective view of the implantable device assembly shown in FIG. 4 with retaining members;

FIG. 6 is a side view of the bone fusion cage assembly of FIG. 1 in an expanded state and carried by a compression tool;

FIG. 7 is a side view of the bone fusion cage assembly of FIG. 1 in a compressed state and carried by a compression tool;

FIG. 8 is a perspective view of the bone fusion cage assembly of FIG. 1 in an uncompressed state carried by another example compression tool;

FIG. 9 is a side view of the bone fusion cage assembly and compression tool of FIG. 8;

FIG. 10 is a side view of the bone fusion cage assembly of FIG. 1 positioned between two bone members;

FIG. 11 is a side view of a bone fusion cage assembly in accordance with the present disclosure, wherein the bone fusion cage assembly is in a compressed state;

FIG. 12 is a cross-sectional view of the bone fusion cage assembly of FIG. 11;

FIG. 13 is a perspective view of the bone fusion cage assembly of FIG. 11 with pivot members removed;

FIG. 14 is a side view of the bone fusion cage assembly of FIG. 13;

FIG. 15 is a side view of the bone fusion cage assembly of FIG. 11 in a partially expanded state;

FIG. 16 is a cross-sectional view of the bone fusion cage assembly of FIG. 15;

FIG. 17 is a side view of the bone fusion cage assembly of FIG. 11 in a partially expanded state with the pivot members arranged non-parallel relative to each other;

FIG. 18 is a cross-sectional view of the bone fusion cage assembly of FIG. 17;

FIG. 19 is a perspective view of the bone fusion cage assembly of FIG. 18 with a fastening member being added to fix a pivot position of one of the pivot members;

FIG. 20 is a perspective view of the bone fusion cage assembly of FIG. 1 and a first mounting plate assembly;

FIG. 21 is a schematic side view of the bone fusion cage assembly and first mounting plate assembly of FIG. 20 mounted to a pair of bone members;

FIG. 22 is a schematic side view of the bone fusion cage assembly and first mounting plate assembly of FIG. 20 mounted to bone members;

FIG. 23 is a perspective view of the bone fusion cage assembly of FIG. 1 and a second mounting plate assembly;

FIG. 24 is a schematic side view of the bone fusion cage assembly of FIG. 1 and a third mounting plate assembly having pivotable plate members;

FIG. 25 is another schematic side view of the bone fusion cage assembly and third mounting plate assembly of FIG. 24;

FIG. 26 is a schematic side view of the bone fusion cage assembly of FIG. 1 and a fourth mounting plate assembly.

### BEST MODE(S) FOR CARRYING OUT THE INVENTION

The technology of present disclosure is directed to bone fusion cage assemblies and related methods. The bone fusion cage assemblies described herein are adapted for insertion into a defect or gap between two surfaces, such as in a gap between adjacent bone structures. The bone fusion cage assemblies are typically adapted to automatically expand to the necessary height dictated by the size of the gap. The bone fusion cage assemblies may include features that provide automatic conforming of the bone fusion cage assembly to opposing asymmetrical surfaces of the gap. In at least one example, the bone fusion cage assembly includes pivotal or swiveling end pieces to conform to such asymmetrical (i.e., non-parallel) surfaces of the gap. Such automatic conforming to the asymmetrical surfaces of the gap provides at least some self-alignment of the bone fusion cage assembly relative to the surfaces (i.e., tissue or bone surfaces) that define the gap.

The gap into which the bone fusion cage assembly is inserted may be defined between soft tissue structures, between hard surface structures such as bone, or between a hard surface structure and a soft tissue structure. The gap may be located at any area of the body such as, for example, inside or outside the spine between adjacent vertebrae. An example gap treated by the bone fusion cage assemblies described herein is a corpectomy defect encountered in spinal fusion surgery.

The bone fusion cage assembly is configured to automatically expand from a compressed state to an expanded state when released. The bone fusion cage assembly may be moved into the compressed state using a compression device such as a scissor-type tool or cranking tool. In at least one construction, the bone fusion cage assembly includes a biasing number positioned internal a housing, wherein the housing includes at least two housing portions that are movable toward and away from each other. The compression device compresses the biasing member by moving the housing portions relative to each other. The compressed state of the bone fusion cage assembly may be maintained using a release mechanism or other device. Actuating the release mechanism permits the housing portions to move into an expanded state upon application of a biasing force from the biasing member to fill the gap into which the bone fusion cage assembly is inserted. After the bone fusion cage assembly is expanded into the expanded state, a fastener or other retaining member may be used to fix the housing portions relative to each other. Thereafter, a plate structure mounted to the bone fusion cage assembly may be attached to the structure that defines the gap. The plate structure may be mounted to the bone fusion cage assembly prior to or after expanding the bone fusion cage assembly in the gap.

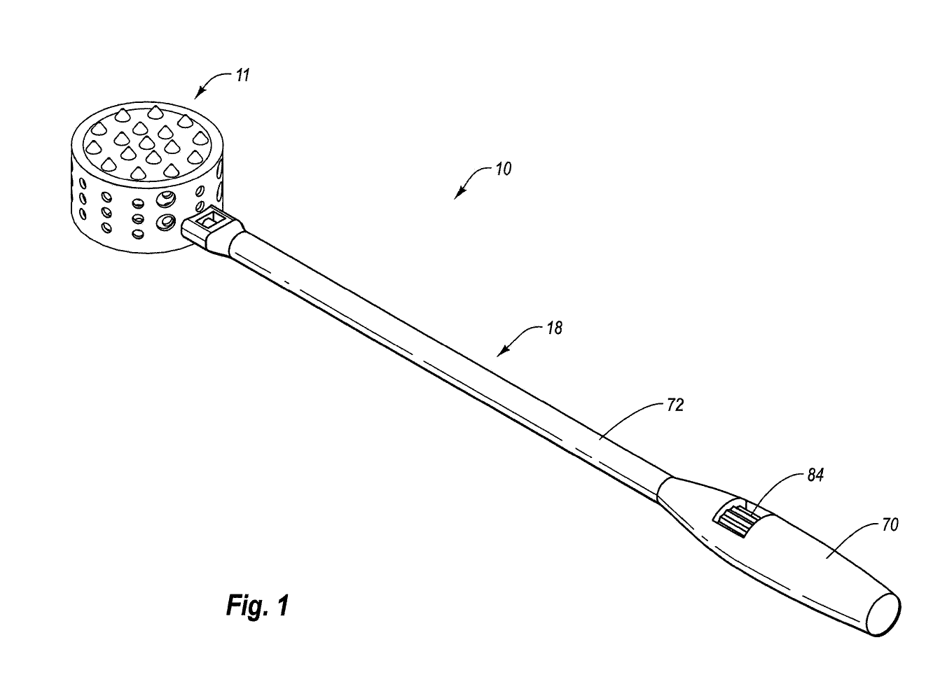

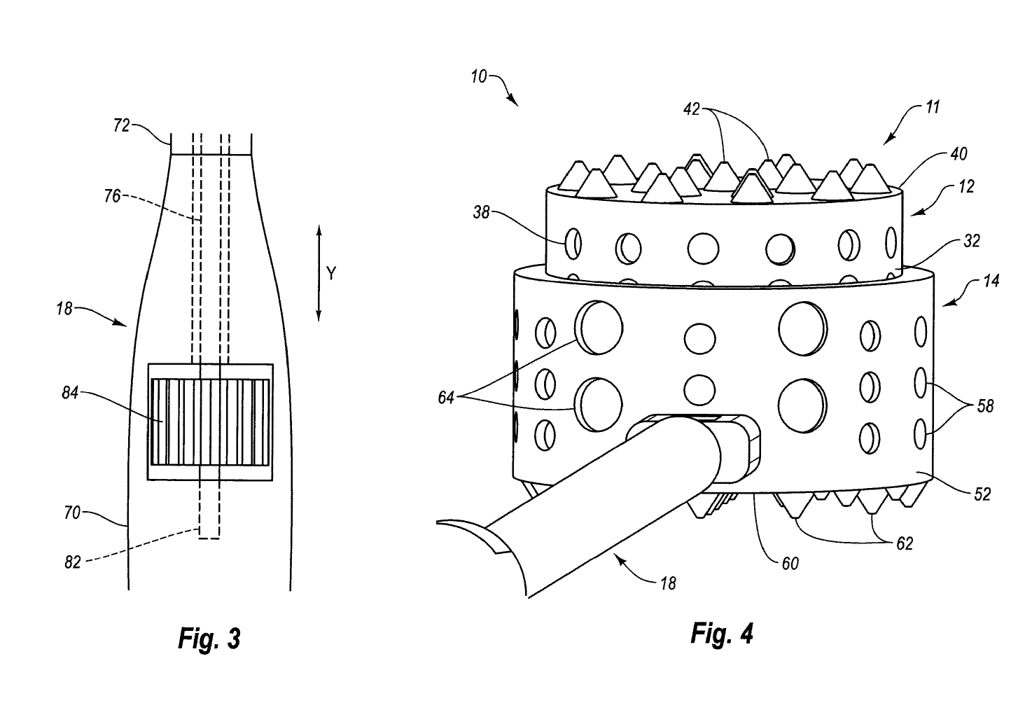

Referring now to FIGS. 1-5, an example of an implantable device assembly 10 having a bone fusion cage assembly 11 is shown and described. The bone fusion cage assembly 11 is coupled to an insertion tool 18. The insertion tool 18 may be used to deliver the bone fusion cage assembly 11 into a gap between opposing structures or surfaces (i.e., between two vertebrae). The insertion tool 18 may also include features that release the bone fusion cage assembly 11 for automatic expansion from a compressed state into an expanded state within the gap.

The bone fusion cage assembly 11 includes first and second housing members 12, 14, and a biasing member 16. The first housing member 12 is at least partially inserted within a cavity defined by the second housing member 14. The biasing member 16 is positioned within at least one of the housing members 12, 14. The positioning of housing members 12, 14 is exemplary and alternative interleaving of the members is possible.

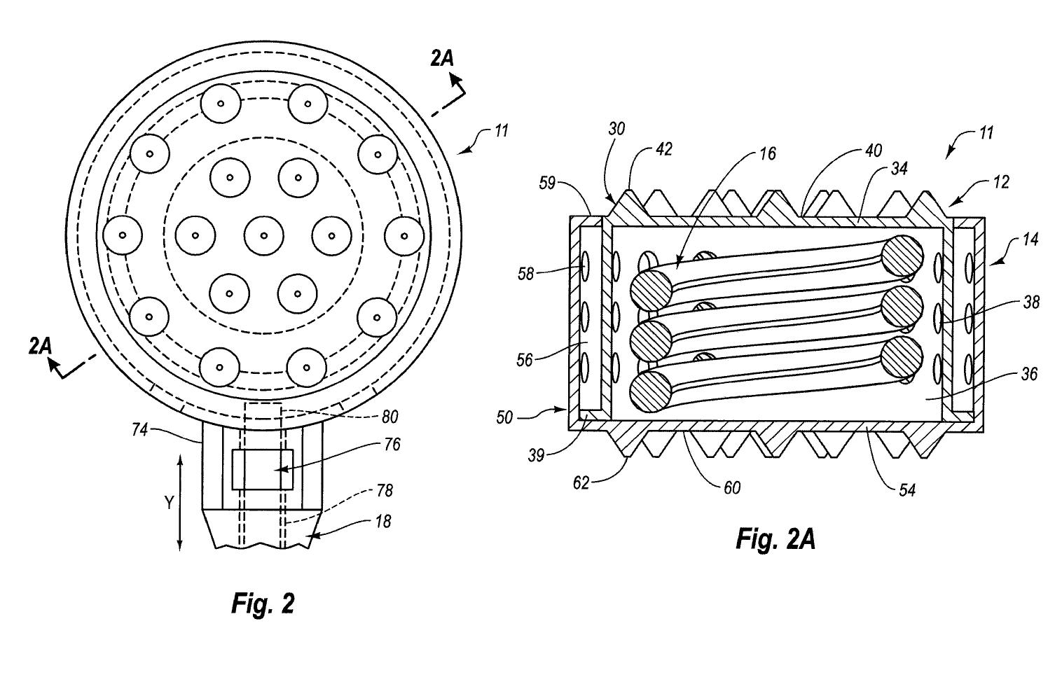

Referring now to FIGS. 2A, 4, 4A, and 5, the bone fusion cage assembly 11 is described in more detail. The first housing member 12 includes a base 30 having a perimeter wall 32, a top wall 34, a hollow cavity 36 defined therein, a plurality of perforations 38 formed in the peripheral wall 32, and a lip feature 39. The perimeter wall 32 has a maximum dimension or diameter D₁ defined along an outer surface of the perimeter wall 32. Typically, the lip feature 39 extends radially outward from the perimeter wall 32. The lip feature 39 provides an engagement surface for contact with a portion of the second housing member to help retain the first and second housing members 12, 14 together.

The top wall 34 defines a contact surface 40. A plurality of spikes or engagement members 42 may be positioned along the contact surface 40. The spikes 42 may provide improved engagement with a surface of the structure that defines the gap into which the bone fusion cage assembly 11 is inserted. While shown as spikes 42, engagement members 42 may be surface texturing, roughening or the like. In some arrangements, the spikes 42 are configured to at least partially penetrate a surface of the structure that defines the gap.

The second housing member 14 includes a base 50 having a perimeter wall 52, a top wall 54, a hollow cavity 56 defined therein, a plurality of perforations 58 formed in the perimeter wall 52, and a lip member 59 that extends radially inward from the peripheral wall 52. The perimeter wall 52 may have a maximum inner dimension or diameter D₂ along an inner surface of the perimeter wall 52. Typically, the diameter D₂ is greater than the diameter D₁ of the first housing member 12.

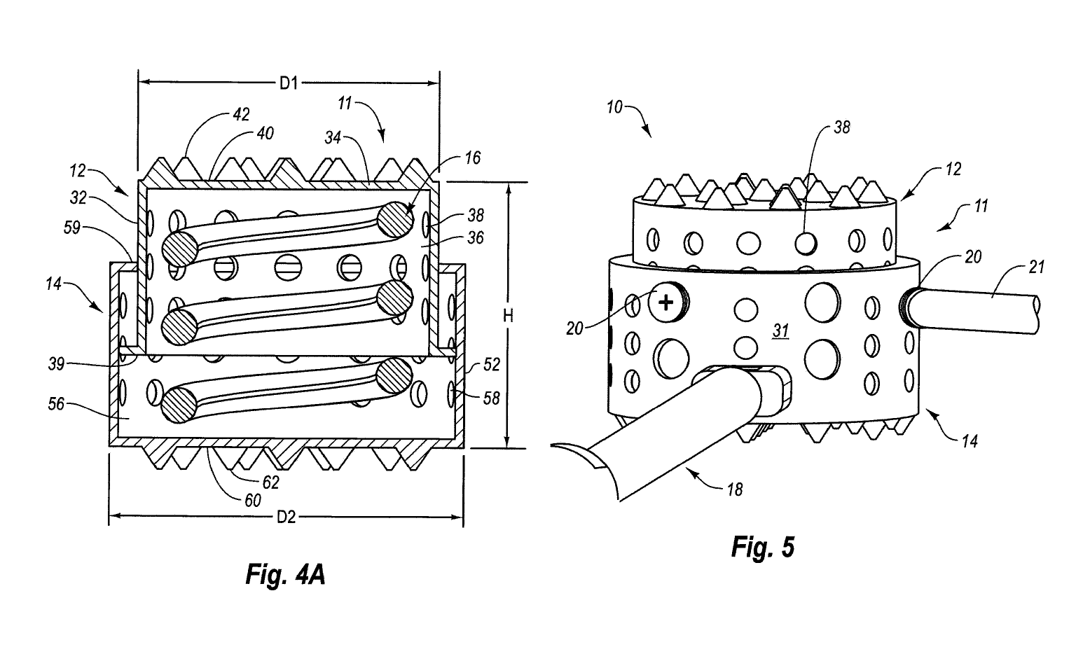

The lip 59 is arranged and configured to engage the lip feature 39 of the first housing member 12 to limit separation of the first and second housing members 12, 14 from each other after assembly of the first and second housing members 12, 14. In at least some arrangements, one or both of the first and second housing members 12, 14 include flexible or deformable portions that permit insertion of the first housing member 12 into the second housing member 14 so that the lip feature 39 moves axially past the lip feature 59. In some arrangements, one or both of the lip features 39, 59 includes flexible or deformable properties that permit the lip 39 to move past the lip 59 while inserting the housing member 12 into the second housing member 14. In other arrangements, one of the lip features 39, 59 is added after at least partial insertion of the first housing member 12 into the second housing member 14. Other features besides the lip features 39, 59 may be used to limit separation of the first and second housing members 12, 14 in the axial direction while permitting at least some axial movement between the first and second housing members 12, 14 relative to each other (e.g., movement between the contracted and expanded states shown in FIGS. 2A and 4A, respectfully).

The top wall 54 defines a contact surface 60. The contact surface 60 is arranged and configured to engage a surface of the structure that defines the gap into which the bone fusion cage assembly 11 is inserted. A plurality of spikes or other engagement member 62 may be included along the contact surface 60 to improve contact with the surfaces that define the gap. While shown as spikes 42, engagement members 62 may be surface texturing, roughening, or the like. In some arrangements, the spikes 62 may be configured to penetrate the surface of the structure that defines the gap.

A plurality of fastener apertures 64 may be defined in the perimeter wall 52. The fastener apertures 64 are sized to receive a fastener or other engagement device such as, for example, a set screw. In one arrangement, a set screw is advanced through the fastener aperture 64 and into engagement, such as, for example, via a frictional engagement, with the perimeter wall 32 of the first housing member 12 to fix or otherwise lock a position of the first housing member 12 relative to the second housing member 14. In other arrangements, the first housing member 12 also includes a plurality of fastener apertures (not shown), and the set screw is advanced through one of the fastener apertures 64 of the second housing member 14 and into one of the fastener apertures of the first housing member 12 to fix or otherwise lock a position of the first housing member 12 relative to the second housing member 14.

The second housing member 14 may include a plurality of fastener apertures 64 spaced around a perimeter of the peripheral wall 52 and along a length of the perimeter wall between the top wall 54 and the lip 59. Providing a plurality of fastener apertures 64 may increase the number of options the operator has for locking the first housing member 12 relative to the second housing member 14 when the first and second housing members 12, 14 are at various relative axial positions.

FIG. 5 illustrates insertion of a pair of set screws 20 or other retaining member 20 into the second housing member 14 using a fastener driver 21. In at least one example, the fastener aperture 64 is positioned along a front side 31 of the second housing member 14 to be accessible by the fastener driver 21 at a location adjacent to the point of connection between the insertion tool 18 and the bone fusion cage assembly 11. Providing the operator with the ability to adjust and fix a height H (see FIG. 4A) of the bone fusion cage assembly 11 defined between contact surfaces 40, 60 may make use of the bone fusion cage assembly 11 easier for different gap sizes.

Typically, the spring 16 has properties that apply a biasing force for any given height H possible for the bone fusion cage assembly 11. In some arrangements, the spring 16 is configured to apply a biasing force for only a certain range of height H, such as up to a height H that is the maximum height of a gap into which bone fusion cage assembly 11 is inserted.

The spring 16 is shown as a single spring operable within the cavities 36, 56 of the first and second housing members 12, 14. Many other arrangements and configurations for the spring 16 are possible while providing the same or similar function as described above. For example, a single spring 16 may be positioned on an outer surface of one or both of the perimeter walls 32, 52 of the first and second housing members 12, 14. In other arrangements, two or more spring members may be operable within the cavities 36, 56, or outside either one of the cavities 36, 56. Further, the spring members may be configured as expansion springs rather than compression springs depending on the orientation of the springs relative to the housing members.

Typically, the biasing member 16 and the first and second housing members 12, 14 comprise a biocompatible material such as titanium, PEEK, or Nitinol. Other material, biocompatible metals, alloys, plastics, ceramics, and composites are possible.

The perforations 38, 58 of the first and second housing members 12, 14 may be structured to permit the growth of body tissue therethrough. In one example, new bone tissue growth may extend through the perforation 38, 58 and into the cavities 36, 56. In at least one example, a plurality or mass of additional growth material, such as bone chips, may be positioned within at least one of the hollow cavities 36, 56 prior to inserting the bone fusion cage assembly 11 within the gap. Cavities 36, 56 also may be packed with osteogenic cells to facilitate bone growth. Osteogenic cells include, for example, bone morphogenetic proteins (BMP) or the like. The additional growth material within the first and second housing members 12, 14 may promote faster growth of tissue through the perforations 38, 58, and improve fusion and acceptance of the bone fusion cage assembly 11 by the patient's body.

The insertion tool 18 may be used to insert the bone fusion cage assembly 11 into a gap and then release the bone fusion cage assembly 11 for automatic expansion. The insertion tool 18 includes a handle 70, a shaft 72, a rod 76, having a distal end 80 and a proximate end 82, and an actuator 84. Shaft 72 has a connector end 74 with a plurality of connection features used to mount the insertion tool 18 to the bone fusion cage assembly 11. Shaft 72 also defines a lumen 78 through which the rod 76 extends.

The rod 76 extends distally from the connection end 74 of the shaft 72 for engagement with a portion of the bone fusion cage assembly 11. Typically, a portion of the shaft 72 is inserted through an aperture defined in the perimeter wall 52 of the second housing member 14 and into engagement with first housing member 12 (i.e., by insertion through another aperture arranged in the perimeter wall 32 of the first housing member 12).

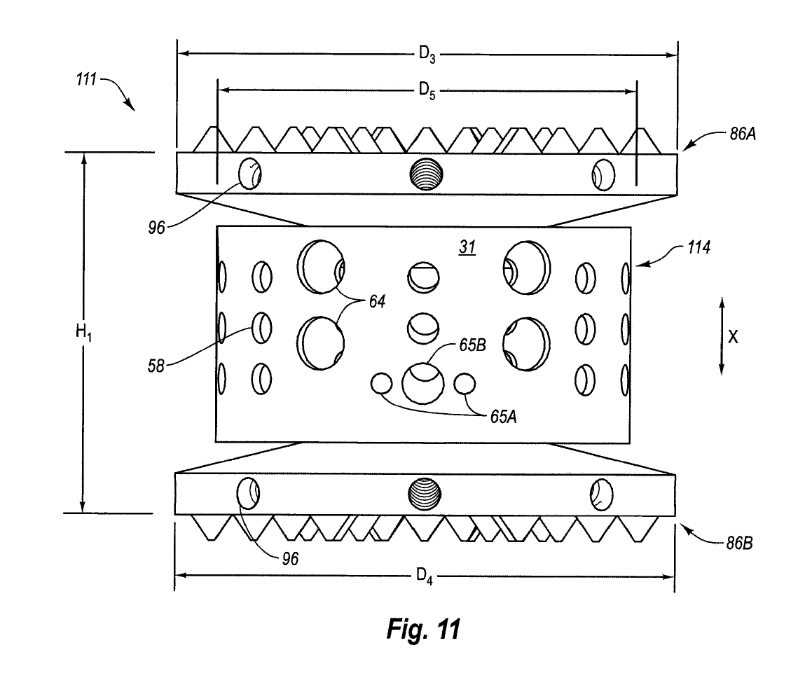

Referring to FIG. 11, an example set of insertion tool aperture 65A, 65B are shown positioned along a front surface 31 of a second housing member 114. Similar apertures may be used on second housing member 14. The first insertion tool apertures 65A are sized and arranged to receive features of the connector end 74 of the insertion tool 18. The second insertion tool aperture 65B is arranged for passage of the shaft 72 through the second housing member 14 where the shaft 72 engages the first housing member 12.

The actuator 84 is shown positioned at least partially within the handle 70. The actuator 84 may include a gear assembly or other features that operates to move the shaft 72 in a release direction Y to move the shaft 72 out of engagement with the first housing member 12 (see FIG. 2). In one example, the actuator 84 includes a roller that rotates about an axis that is arranged parallel with the shaft 72, wherein rotation of the actuator advances the shaft 72 in a proximal direction. In other arrangements, the actuator 84 includes a thumb actuated slide and the handle 70 includes a track arranged parallel with the shaft 72, wherein advancing the actuator 84 in the proximal direction moves the shaft 72 in the release direction Y. Many other configurations are possible for the actuator 84.

Other devices, instruments, and methods may be used to secure the first and second housing members 12, 14 together in a compressed state as shown in FIG. 1 and then release the first and second housing members 12, 14 from each other to permit relative movement of the first and second housing members 12, 14 away from each other into an expanded state.

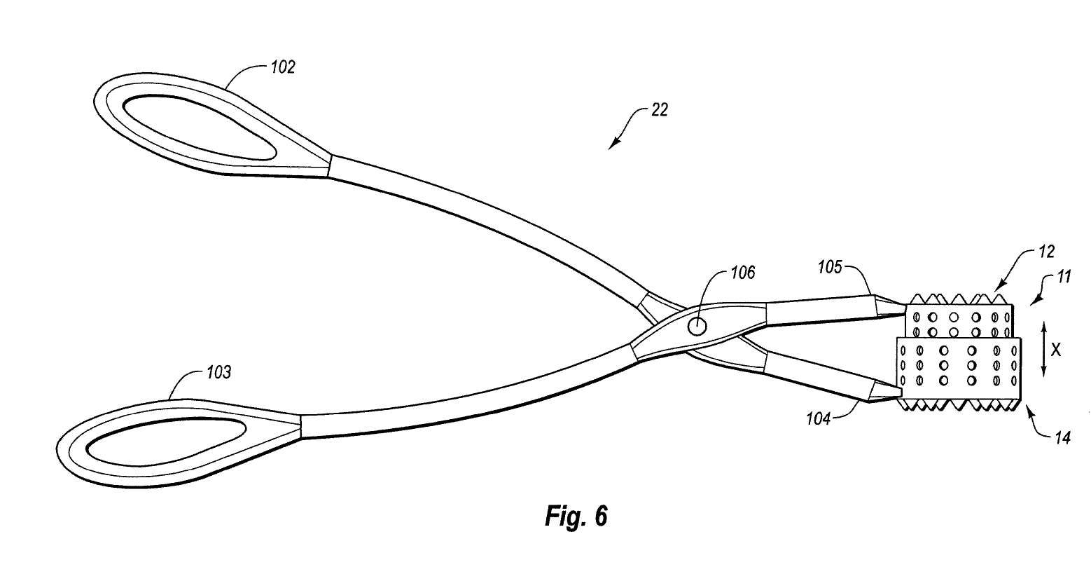

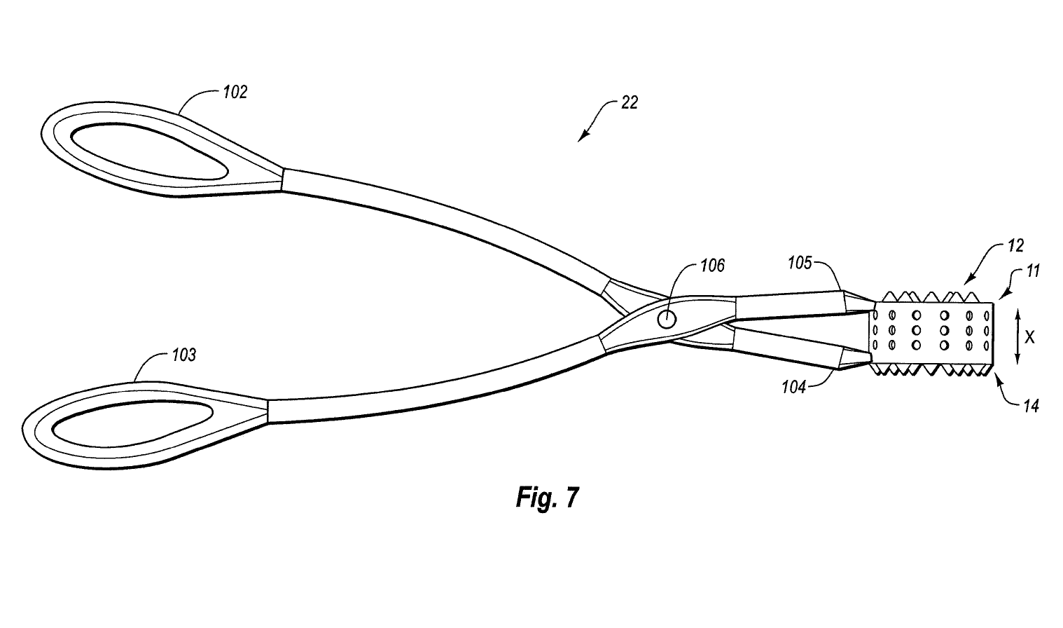

Referring to FIGS. 6 and 7, an example compression tool 22 is shown and described. The compression tool 22 includes first and second gripping members 102, 103, first and second contact members 104, 105, and a pivot point 106. The first and second contact members 104, 105 are arranged in engagement with the second and first housing members, 14, 12, respectively. Movement of the first and second gripping members 102, 103 toward each other moves the first housing member 12 towards the second housing member 14 in the direction X from the uncompressed state shown in FIG. 6 to the compressed state shown in FIG. 7. Typically, the insertion tool 18 is mounted to the second housing member 14 and the shaft 72 is advanced through the second housing member 14 and into engagement with the first housing member 12 to maintain the compressed state shown in FIG. 7.

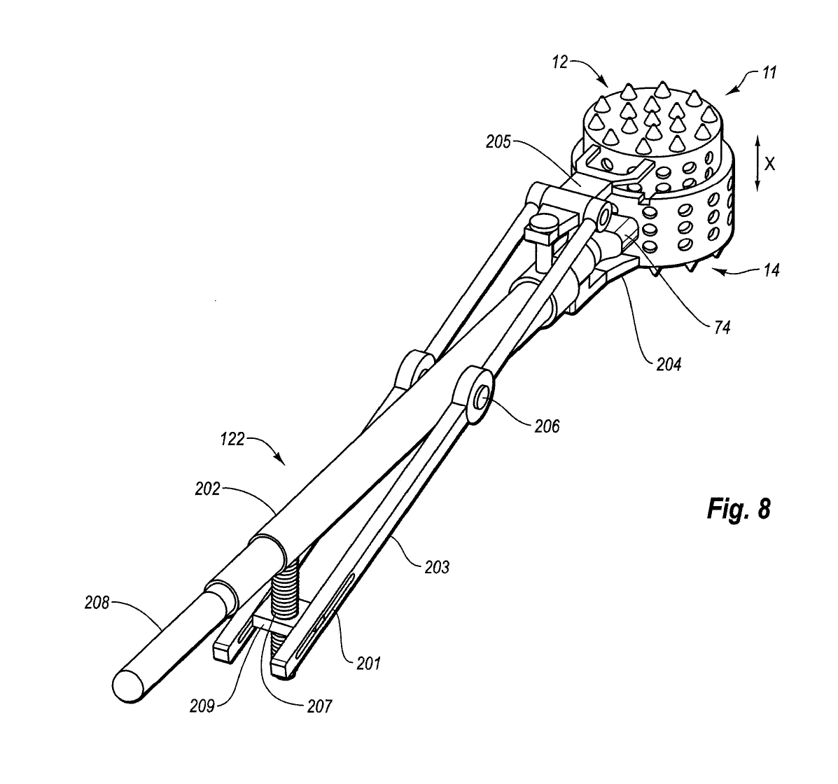

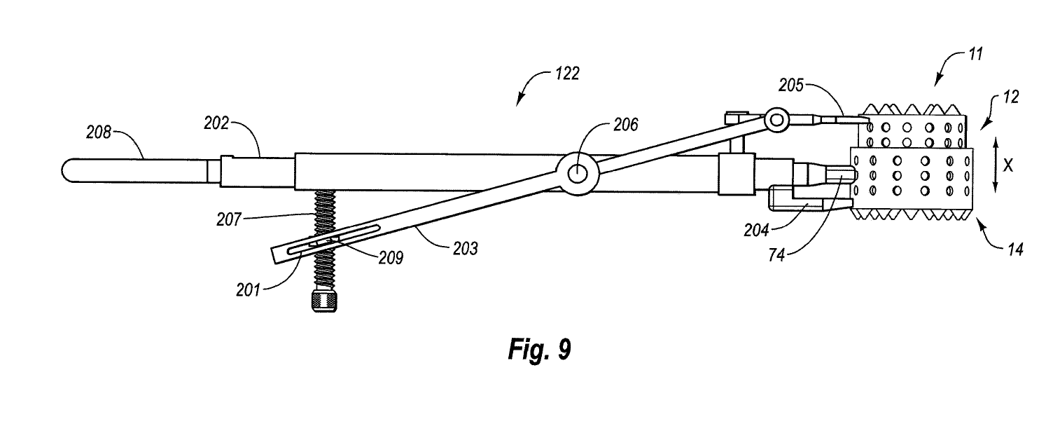

FIGS. 8 and 9 illustrate another example compression tool 122 that is configured to compress the bone fusion cage assembly 11 from an expanded state (e.g., see FIG. 6) to a compressed state (e.g., see FIG. 7). The compression tool 122 includes first and second lever ends 202, 203, first and second contact members 204, 205, a pivot point 206, an actuator 208, and a threaded rider 209. Turning the bolt 207 causes the threaded rider 209 and the constrained second lever arm 203 to travel toward or away from the first lever end 202 and compresses the spring 16 or allows the spring 16 to expand.

A portion of the compression tool 122 can move within a slot 201 defined in the second lever member 203 to permit relative movement of the lever members 202, 203. The compression tool 122 also may include features of the insert tool 18, such as the rod 76 (not shown) and the connection end 74 connected to the second housing member 14. The compression tool 122 compresses the bone fusion cage 11 into a compressed state prepared for insertion into a gap. The compression tool 122 also may release the first and second housing members 12, 14 relative to each other after the bone fusion cage assembly 11 is positioned in the gap. The compression tool 122 may further provide recompression of the bone fusion cage assembly 11 into a compressed or semi-compressed state after being positioned and expanded in the gap. Recompression of the bone fusion cage assembly 11 after being expanded in a gap may be required when repositioning of the bone fusion cage assembly 11 is needed.

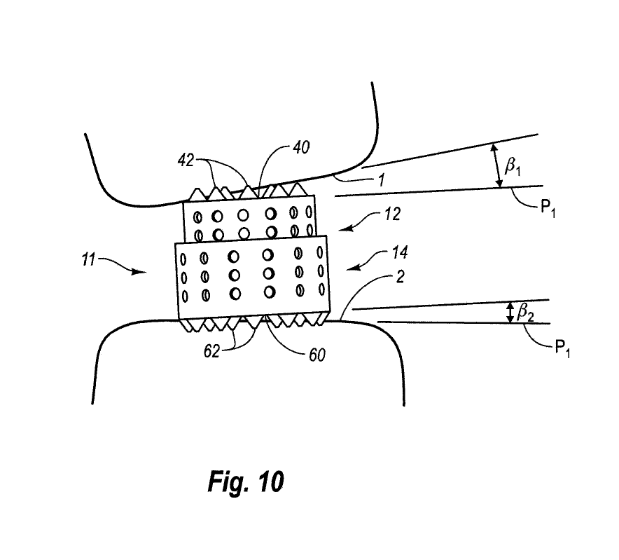

FIG. 10 illustrates insertion of the bone fusion cage assembly 11 positioned within a gap defined between first and second surfaces 1, 2. Surface 1 is arranged in an angle β₁ relative to a horizontal plane P₁. The second surface 2 is arranged at second angle β₂ relative to the plane P₁. The surfaces 1, 2 are not parallel in FIG. 10. The contact surfaces 40, 60 of the first and second housing members 12, 14 respectively may be arranged generally parallel to each other. In some arrangements, the bone fusion cage assembly 11 may be configured to provide some side-to-side lateral movement of the first and second housing members 12, 14 relative to each other to provide a slightly non-parallel arrangement of the contact surfaces 40, 60. The maximum non-parallel angle between the contact surfaces 40, 60 may be less than the non-parallel angled relationship between the first and second surfaces 1, 2 (i.e., β₁+β₂).

Referring now to FIGS. 11-19, another exemplary bone fusion cage assembly 111 is shown and described. The bone fusion cage assembly 111 may be better suited to inner face with surfaces 1, 2 shown in FIG. 10 when the surfaces 1, 2 are angled at angles β₁, β₂ relative to the horizontal plane P₁.

The bone fusion cage assembly 111 includes first and second housing numbers 112, 114. The first housing member 112 includes a base 30 and pivot member 86A. The second housing member 114 includes a base 50 and a pivot member 86B. The pivot members 86A, 86B define contact surfaces 140, 160. Typically, the pivot members 86A, 86B are configured to rotate or pivot relative to the bases 30, 50 sufficient to arrange the contact surfaces 140, 160 at non-parallel orientations relative to each other.

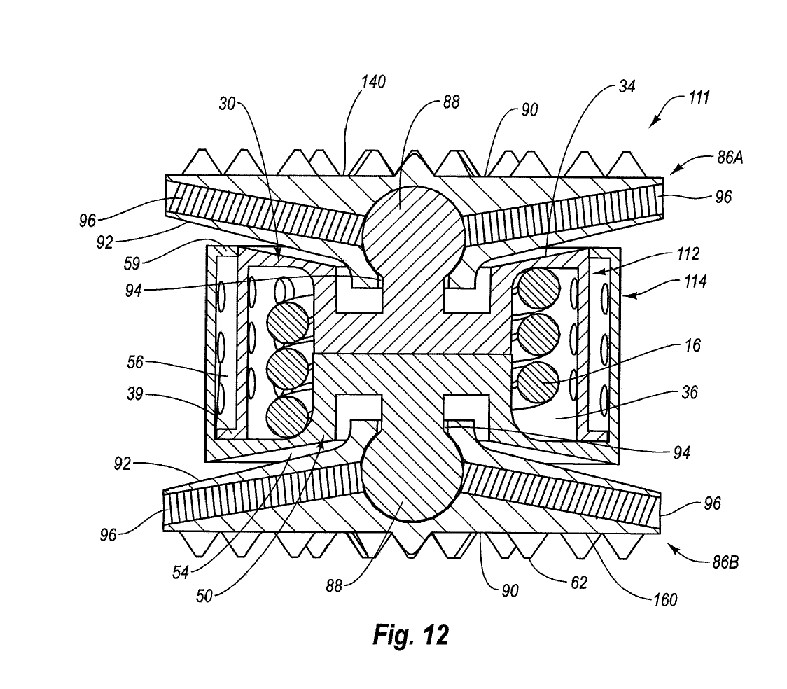

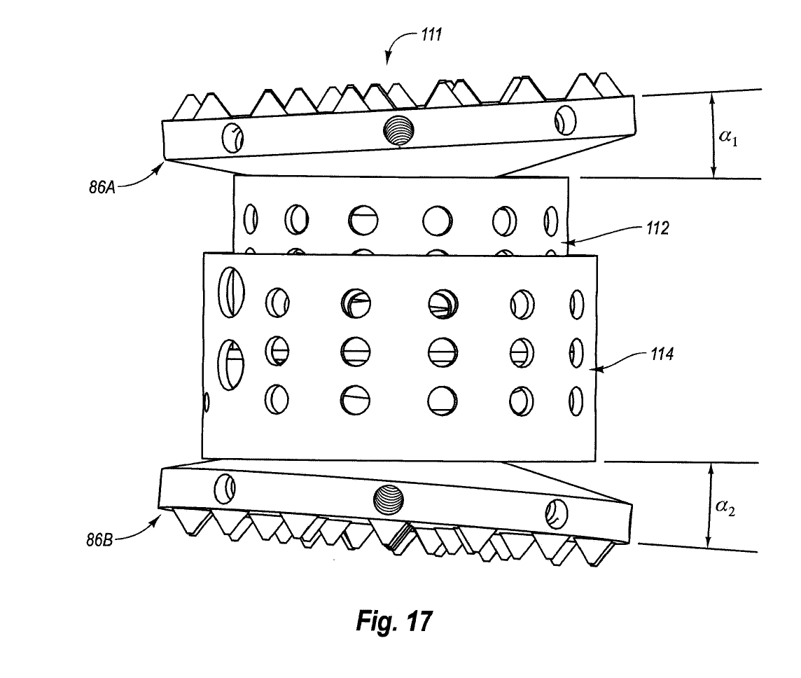

The pivot member 86A includes a top surface 90, a bottom surface 92, a socket feature 94 defined in the bottom surface 92, and at least one fastener aperture 96. The top surface 90 defines the contact surface 140 for the first housing member 112. A plurality of spikes 42 are included on the contact surface 140. The socket feature 94 is configured to engage with a ball structure 88 that is mounted to a top wall 34 of the base 30. In other arrangements, the position of ball 88 and socket 94 may be reversed. The inner face between the ball 88 and socket 94 provide pivotal movement of the pivot member 86A relative to the base 30 through a tilt angle α₁ (see FIG. 17). The top wall 34 may include a recessed surface that mirrors the shape of bottom surface 92 (see FIG. 12).

The pivot member 86A may include a maximum diameter or dimension D₃. The diameter D₃ may be greater than a maximum outer diameter or dimension D₅ of the second housing member 114 (see FIG. 11). Other features of the first housing member 112 may be the same or similar to the first housing member 12 described above. For example, the first housing member 112 may include a hollow cavity 36, a plurality of perforations 38, and a lip feature 39.

The pivot member 86B includes a top surface 90, a bottom surface 92, a socket feature 94 defined in the bottom surface 92, and at least one fastener aperture 96. The top surface 90 defines a contact surface 160. A plurality of spikes 62 is included on the contact surface 160. The socket feature 94 is configured to engage a ball structure 88 that is mounted to a top wall 54 of the base 50. In some arrangements, the position of socket 94 and ball 88 may be reversed. The ball and socket arrangement of second housing member 114 provides pivotal movement of the pivot member 86B relative to the base 50 of the second housing member 114. Typically, the pivot member 86B can move through a tilt angle α₂ relative to the horizontal plane, such as a plane defined by the top surface 54 (see FIG. 17). The top wall 54 may define a recess that mirrors a shape of the bottom side 92.

The ball and socket arrangement of the first and second housing members 112, 114 may be replaced with other pivot or hinged structures. In one example, the ball and socket arrangement is replaced with a hinge member.

The pivot member 86B may have a maximum diameter or dimension D₄. The dimension D₄ may be equal to the dimension D₃ of the pivot member 86A. The dimension D₄ may be greater than the maximum outer diameter or dimension D₅ of the second housing member 114 (see FIG. 11).

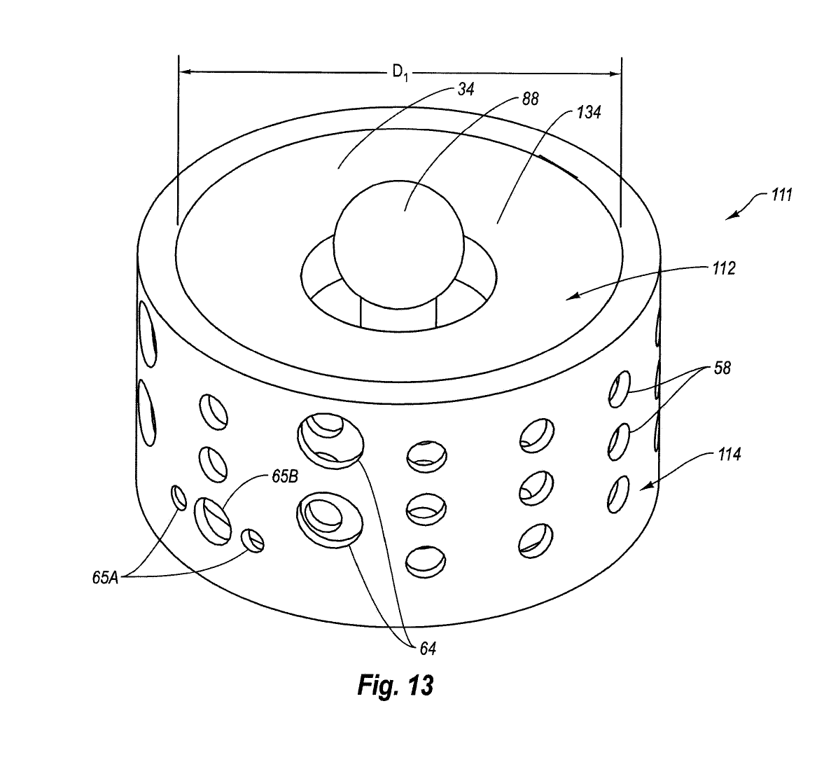

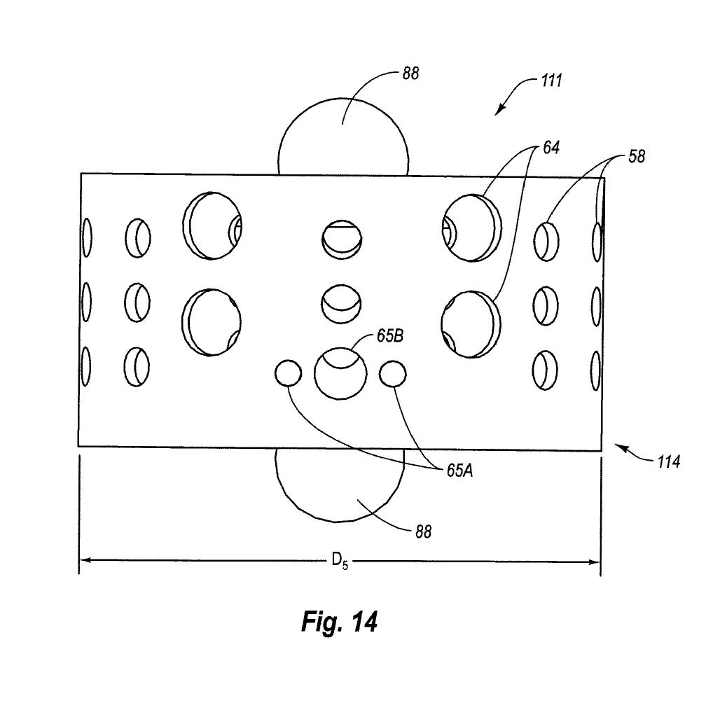

The bone fusion cage assembly 111 includes a biasing member 116 that is operable within the first and second housing members 112, 114 to axially move the first and second housing members 112, 114 away from each other. FIGS. 11 and 12 illustrate the bone fusion cage assembly 111 in a compressed state. The bone fusion cage assembly 111 in FIGS. 11 and 12 has a height H₁ between the contact surfaces 140, 160. The pivot members 86A, 86B are arranged generally parallel with each other with the angles α₁, α₂ being substantially zero. FIGS. 13 and 14 illustrate the bone fusion cage assembly 111 without the pivot members 86A, 86B mounted to the ball features 88.

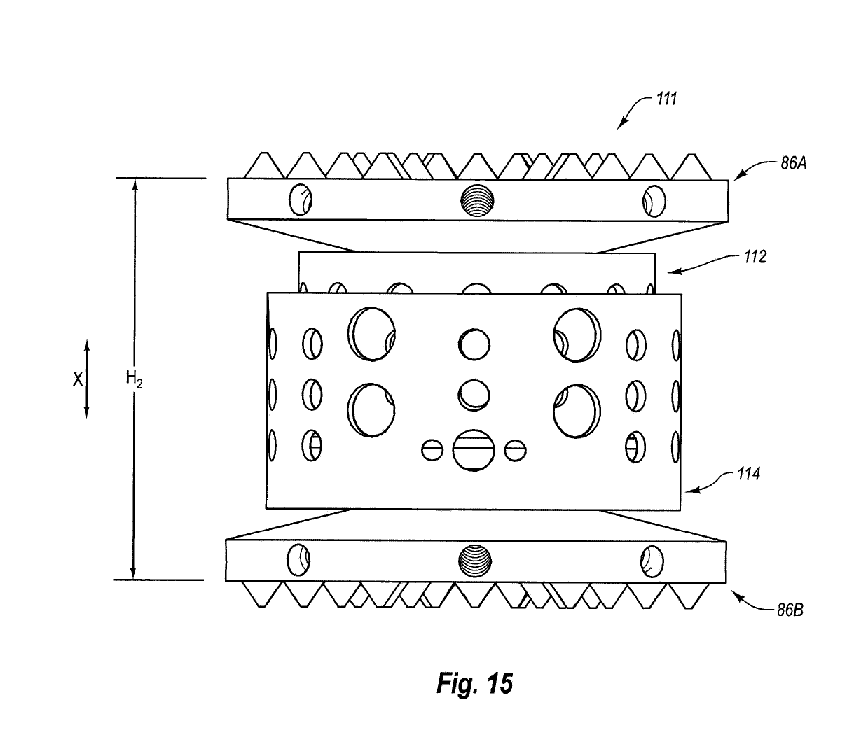

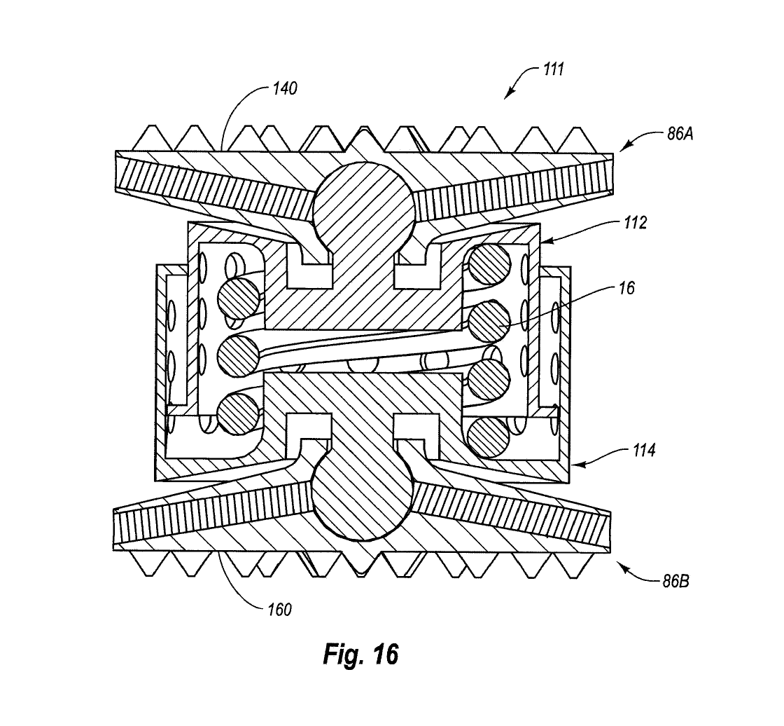

FIGS. 15 and 16 illustrate the bone fusion cage assembly 111 with the first and second housing members 112, 114 released to permit relative axial movement. A distance between the contact surfaces 140, 160 is a height H₂, which is greater than height H_{I}. The contact surfaces 140, 160 are also arranged substantially parallel to each other.

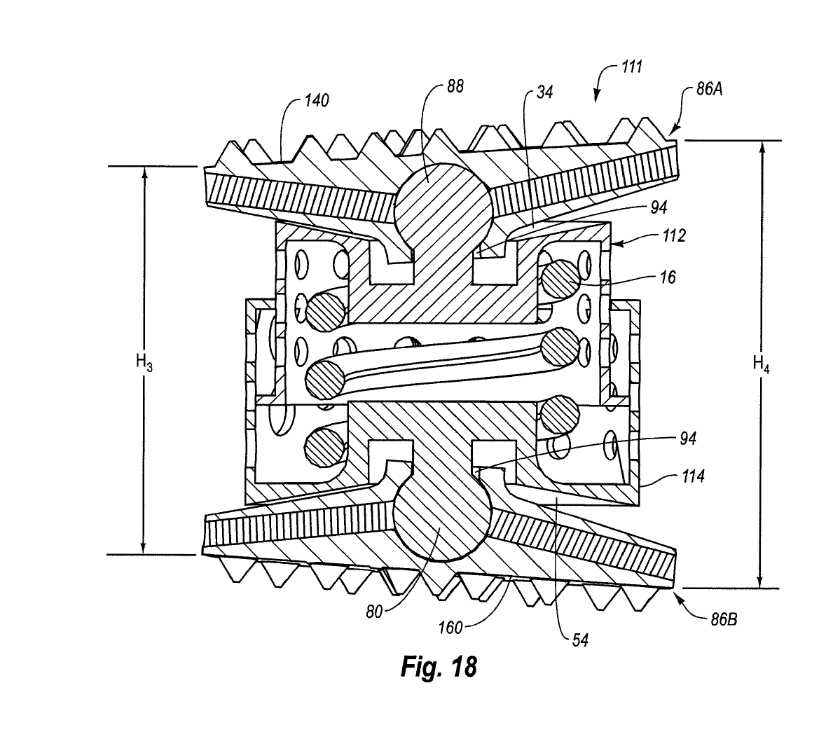

FIGS. 17 and 18 illustrate the bone fusion cage assembly 111 at a further expanded state as compared to the arrangement shown in FIGS. 15 and 16. In FIGS. 17 and 18, the bone fusion cage assembly 111 has a minimum height H₃ defined between contact surfaces 140, 160, and a maximum height H₄ defined between contact surfaces 140, 160. The tilted angles α₁, α₂ are greater than zero so that the contact surfaces 140, 160 are arranged nonparallel relative to each other

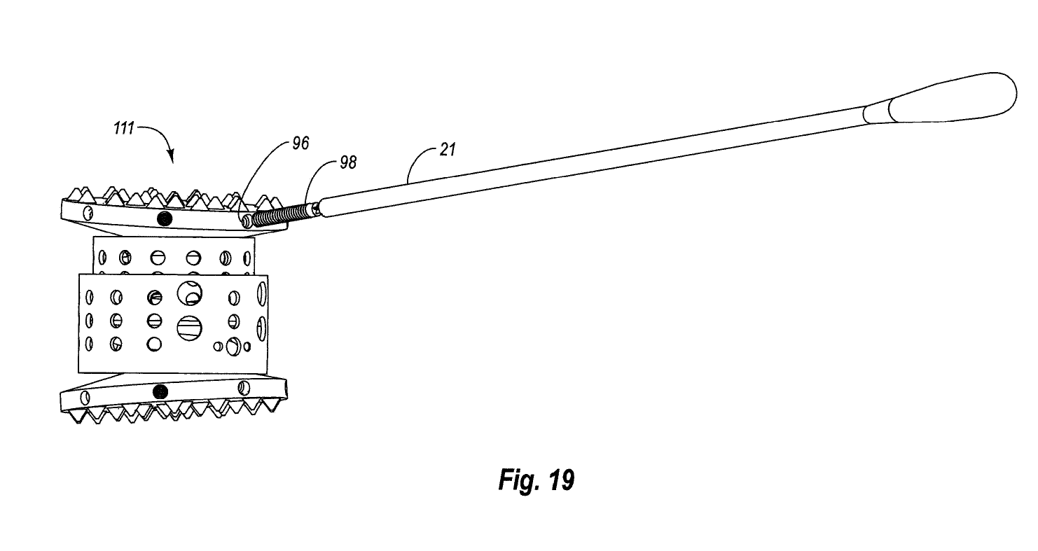

An orientation of the pivot member 86A or 86B relative to the bases 30, 50, respectively, may be fixed using a set screw or other type of fastener. In one example, a set screw is inserted through the fastener aperture 96 and engaged with the ball 88. FIG. 19 illustrates insertion of a set screw 98 through one of the fastener apertures 96 using a fastener driver 21. A plurality of fasteners, such as set screw 98, may be inserted through a plurality of the fastener apertures 96 to provide additional fixation of the pivot members 86A, 86B relative to the bases 30, 50.

The bone fusion cage assembly 111 may be mounted to an insertion tool 18. The insertion tool 18 may be operable with the bone fusion cage assembly 111 similar to operation of the bone fusion cage assembly 11 described above. The insertion tool 18 may be mounted to, for example, the second housing member 114 via the insert tool apertures 65A, 65B shown in FIG. 11.

The bone fusion cage assemblies 11, 111 described above have generally cylindrical constructions with circular cross-sections. Other shapes and sizes are possible for the bone fusion cage assemblies described herein. For example, the cross-sectional shape of the bases 30, 50 may be non-circular in shape such as, for example, hexagonal, triangular or elliptical shaped. The cross-sectional shape of the pivot members 86A, 86B may also have different shapes and sizes instead of the generally circular cross-sectional shape shown in the figures.

The example bone fusion cage assembly described above with reference to FIGS. 1-19 includes first and second housing members wherein the first housing member is insertable into the second housing member. Other housing constructions are possible that provide expansion of the bone fusion cage assembly from a compressed state to an expanded state.

In one example, the bone fusion cage assembly includes three housing members. A first housing member defines a cylindrical core, and the second and third housing members are arranged like cap features that extend over open ends of the first housing member. The biasing member is positioned either inside or outside of the first housing member and operates to move the second and third housing members away from each other in an axial direction along the length of the first housing member. This housing construction can maintain a compressed state in which the second and third housing members are moved toward each other to compress the biasing member by inserting a fastener, such as a set screw, through each of the second and third housing members and into engagement with the first housing member. Releasing the second and third housing members from the first housing member permits automatic expansion of the cage assembly into an expanded state.

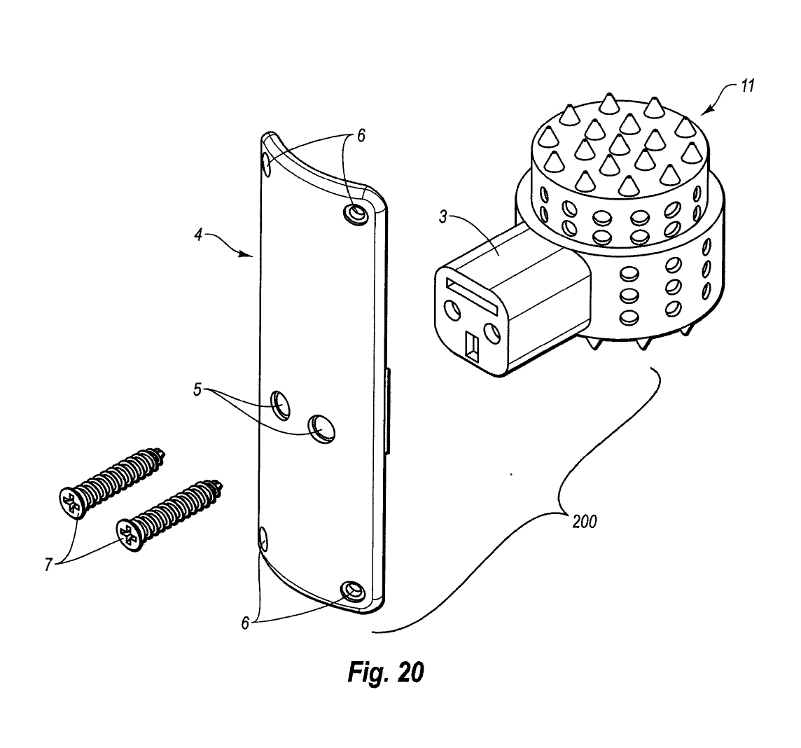

The bone fusion cage assembly described herein may be used in combination with a plate assembly that provides further support to a bone fusion cage assembly and the structure that defines the gap within which the bone fusion cage assembly is positioned. FIGS. 20-22 illustrate an exemplary plate assembly 200 arranged for use with a bone fusion cage assembly 11. The plate assembly 200 includes a connector 3, a plate 4, and a plurality of fasteners 7 used to secure the plate to the connector 3. The connector 3 may include connection features (not shown) similar to those used at the connection end 74 of the insertion tool 18. The connector 3 may have expandable length capabilities for positioning the plate 4 at different distances from the bone fusion cage assembly 11.

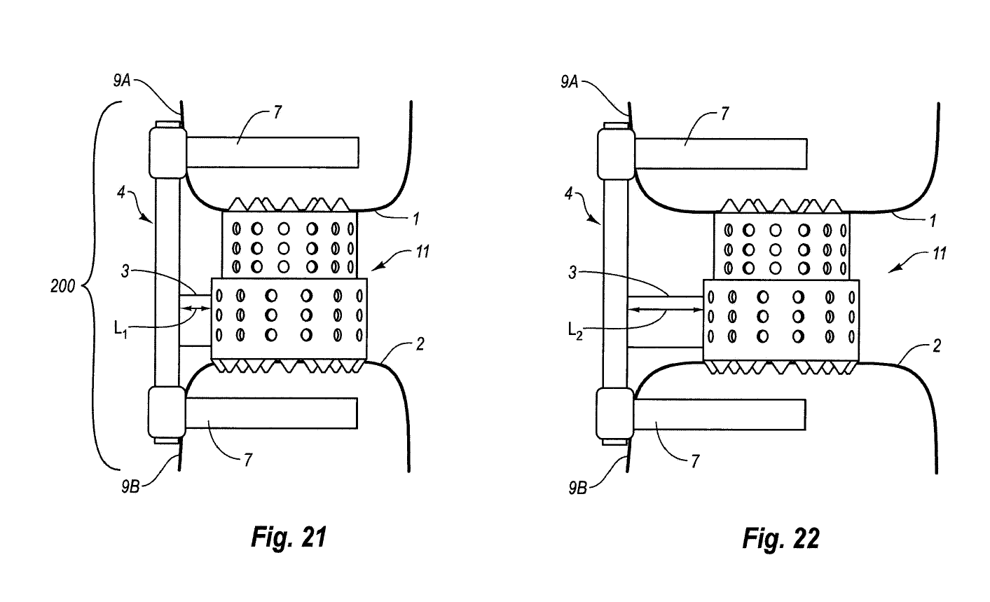

The plate 4 includes at least one connector fastener aperture 5 for securing the plate 4 to the connector 3. The plate 4 may also include a plurality of bone screw apertures 6 through which fasteners (e.g., a bone screw) may be inserted for connecting the plate 4 to the structure that defines the gap within which the bone fusion cage assembly 11 is positioned. In the example shown in FIG. 21, the bone fusion cage assembly is positioned between first and second surfaces 1, 2, the connector 3 has a first length L₁, and the plate 4 is secured to third and fourth surfaces 9A, 9B using a plurality of bone screws 7.

FIG. 22 illustrates another mounted arrangement for the plate assembly 200 wherein the connector 3 has a second length L₂ greater than the first length L₁.

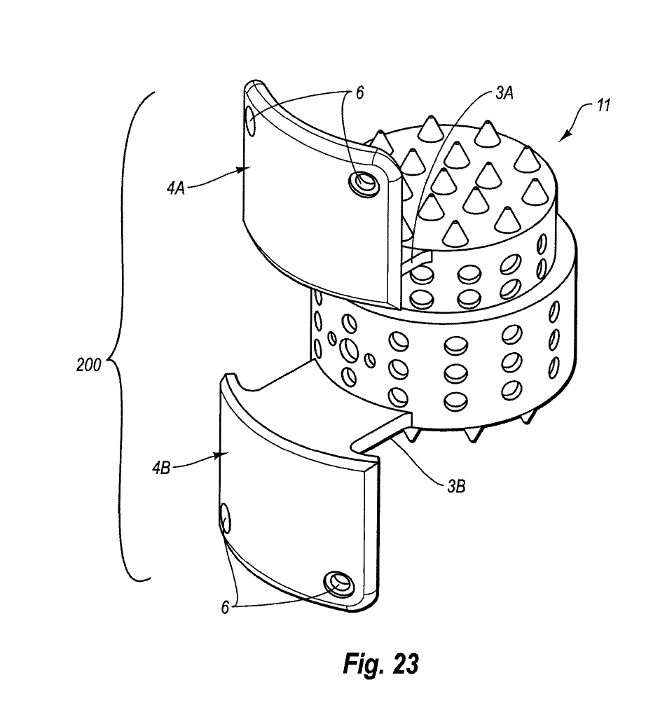

Referring to FIG. 23, another exemplary plate assembly 300 is described for use with the bone fusion cage assembly 11. The plate assembly 300 includes first and second plate portions 4A, 4B connected to the bone fusion cage assembly 11 with individual connectors 3A, 3B, respectively. The plate assembly 300 may be mounted to the bone fusion cage assembly 11 prior to or after expansion of the bone fusion cage assembly 11 within the gap. In some arrangements, at least one of the connectors 3A, 3B is adjustable in length. At least one of the plate portions 4A, 4B may also be adjustable in length. Adjustability of the plate assembly 300 may provide improved contact between portions of the plate assembly 300 and the structure (e.g., vertebrae or other bone structure) to which the plate assembly 300 and bone fusion cage assembly 11 are mounted.

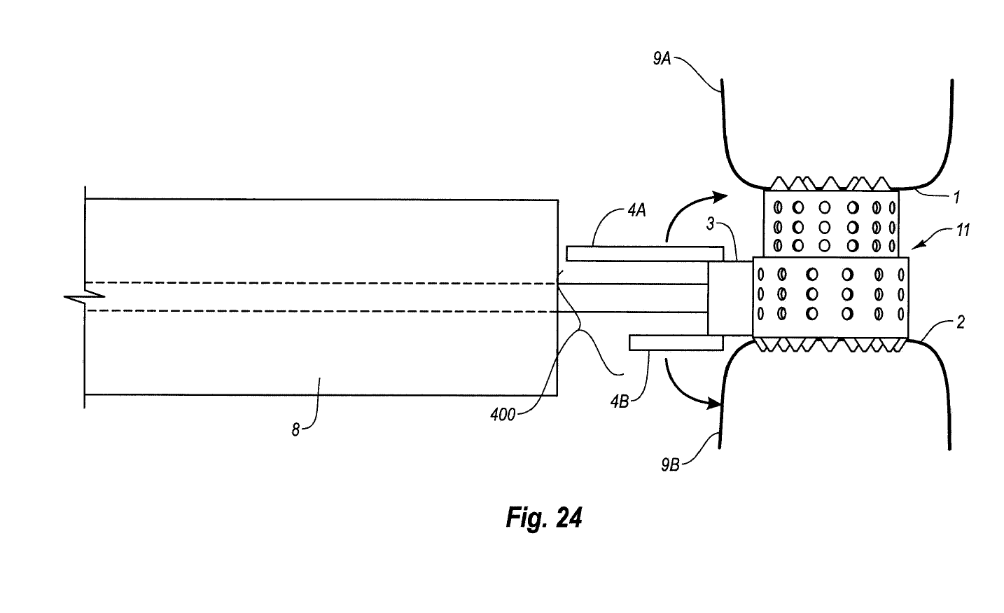

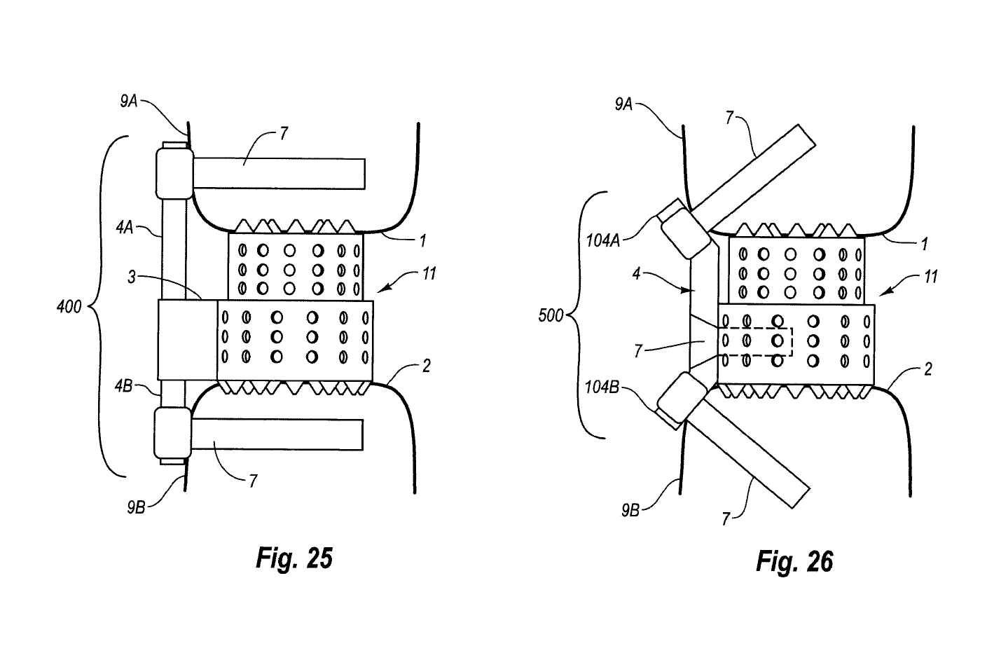

Referring to FIGS. 24 and 25, another example plate assembly 400 is shown and described for use with the bone fusion cage assembly 11. The plate assembly 400 includes first and second plate portions 4A, 4B that are rotatable relative to the connector 3. In FIG. 4, the plate portions 4A, 4B are shown arranged generally parallel with the direction of insertion of the bone fusion cage assembly 11 into the gap defined between surfaces 1, 2. The plate portions 4A, 4B may be rotated into the orientation shown in FIG. 25 into engagement with surfaces 9A, 9B, respectively, after the bone fusion cage assembly 11 has been inserted into the gap and expanded into contact with surfaces 1, 2. Thereafter, the plate portions 4A, 4B may be mounted to the surfaces 9A, 9B using fasteners, such as screws 7.

The plate assembly 400 may be well suited for providing improved visual inspection of the surfaces 1, 2, 9A, 9B by the operator while inserting the bone fusion cage assembly 11 into the gap. In at least one example, the generally parallel arrangement of the plate portions 4A, 4B shown in FIG. 24 provides improved ease in inserting the plate assembly 400 using an endoscope 8 or other insertion device. The rotatable features of the plate portions 4A, 4B may provide improved alignment of and contact between the plate portions 4A, 4B with surfaces 9A, 9B.

The pivotal motion of the plate portions 4A, 4B may be provided by a hinged connection to the connector 3. In other arrangements, portions of the connector 3 or the plate portions 4A, 4B may be flexible or deformable to provide the rotating motion. In still other examples, the plate portions 4A, 4B may be mounted to the connector 3 after the bone fusion cage assembly has been positioned in the gap between surfaces 1, 2.

FIG. 26 illustrates another plate assembly 500 for use with a bone fusion cage assembly 11. The plate assembly 500 includes a plate 4 having angled portions 104A, 104B. The plate 4 may be directly mounted to the bone fusion cage assembly 11 using, for example, a fastener such as screw 7. Alternatively, a connector such as any one of the connectors 3 described with reference to FIGS. 20, 25 may be used to mount the plate 4 to the bone fusion cage assembly 11. The angled orientation of the portions 104A, 104B can provide improved engagement with angled surfaces that are arranged between surfaces 1, 9A and 2, 9B. Fasteners such as screw 7 may be used to secure the plate 4 to the structures. The screws 7 shown in FIG. 26 are arranged at a diagonal angle relative to the surfaces 1, 2, 9A, 9B. The plate assembly 500 may be mounted to the bone fusion cage assembly 11 prior to or after the insertion of the bone fusion cage assembly 11 in the gap defined between surfaces 1, 2.

While the above figures show a plate extending over one level, one of ordinary skill in the art will recognize on reading the disclosure that the present invention would be useful for multiple level fusions. Moreover, although the stabilization device is depicted extending from a single end of the plate, one of ordinary skill in the art, on reading the disclosure, would understand that the present invention could have stabilization devices extending from multiple connection points, i.e., the superior and inferior direction.

While the invention has been particularly shown and described with reference to embodiments thereof, it will be understood by those skilled in the art that various other changes in the form and details may be made without departing from the spirit and scope of the invention.

We claim:

1. A bone fusion cage assembly, comprising:
a first housing member including:
a first contact surface;
a first hollow cylindrical shaped body having a first closed end;
a first pivot portion defining an angular orientation of the first contact surface relative to the first hollow cylindrical shaped body;
a second housing member including:
a second contact surface;
wherein the second housing member is slidably coupled to the first housing member and the second contact surface faces opposite the first contact surface;
a biasing member positioned within at least a portion of the first hollow cylindrical shaped body and operable to bias the first and second housing members away from each other into an expanded state.

2. The bone fusion cage assembly of claim 1, wherein the first pivot portion is mounted to the first cylindrical shaped body with a ball and socket connection.

3. The bone fusion cage assembly of claim 1, wherein the second housing member includes a second pivot portion and a second hollow cylindrical shaped body having a second closed end, the second pivot portion defining an angular orientation of the second contact surface relative to the second hollow cylindrical shaped body.

4. The bone fusion cage assembly of claim 3, wherein the second pivot portion is mounted to the second hollow cylindrical shaped body with a ball and socket connection.

5. The bone fusion cage assembly of claim 3, wherein the first contact surface is positioned at the first closed end and the second contact surface is positioned at the second closed end.

6. The bone fusion cage assembly of claim 1, wherein at least a portion of the first housing member extends within the second housing member.

7. The bone fusion cage assembly of claim 1, further comprising at least one fastener operable to secure the first and second housing members together in the expanded state.

8. The bone fusion cage assembly of claim 1, further comprising a retaining member configured to hold the first and second housing members in an unexpanded state with the biasing member in a compressed state.

9. The bone fusion cage assembly of claim 8, wherein the retaining member includes a release portion that extends into engagement with the first housing member and into engagement with the second housing member, and retraction of the release portion out of engagement with at least one of the first and second housing members permits the biasing member to move the first and second housing members into the expanded state.

10. The bone fusion cage assembly of claim 1, wherein the first and second contact surfaces each include a plurality of spike members.

11. The bone fusion cage assembly of claim 1, wherein the first and second housing members are configured to provide the first and second contact surfaces in both a parallel orientation and a non-parallel orientation.

12. The bone fusion cage assembly of claim 1, wherein each of the first and second housing members includes a retaining portion that provides sliding movement of the first and second housing members relative to each other while limiting separation of the first and second housing members from each other.

13. An implantable device assembly, comprising:
a bone fusion cage assembly, comprising:
a first housing member defining a first contact surface;
a second housing member defining a second contact surface, the second contact surface facing generally opposite the first contact surface;
a biasing member configured to apply a biasing force to the first and second housing members;
an insertion tool, comprising:
an attachment member configured to releasably mount the insertion tool to the bone fusion cage assembly;
a release member;
an actuator operable to move the release member from a first position in which the first and second housing members are retained in a compressed state relative to each other, to a second position in which the first and second housing members are movable away from each other by application of the biasing force.

14. The implantable device assembly of claim 13, wherein the insertion tool further includes a handle portion, the release member extending from the attachment member to the handle portion, and the actuator being positioned on the handle.

15. The implantable device assembly of claim 13, wherein the actuator includes a gear assembly, and rotation of the actuator causes the release member to move axially away from the bone fusion cage assembly from the first position to the second position.

16. The implantable device assembly of claim 13, wherein the first housing member includes a first base and a first pivot member pivotally mounted to the first base, the first pivot member defining the first contact surface, and the second housing member includes a second base and a second pivot member pivotally mounted to the second base, the second pivot member defining the second contact surface.

17. The implantable device assembly of claim 13, further comprising a plate assembly mounted to the bone fusion cage assembly, the plate assembly includes at least one plate member arranged generally perpendicular to the first and second contact surfaces.

18. The implantable device assembly of claim 13, further comprising a plate assembly mounted to the bone fusion cage assembly, the plate assembly includes at least one plate member that pivots between a first position and a second position relative to the bone fusion cage assembly.

19. A method of operating a bone fusion cage assembly, comprising:
providing a bone fusion cage assembly having first and second housing members and a biasing member operable between the first and second housing members, each of first and second housing members each including a contact surface and a base portion, at least one of the first and second housing members including a pivotal connection of the contact surface to the base portion, the method including:
moving the first and second housing members in a direction toward each other to move the biasing member into an unexpanded state;
retaining the first and second housing members together with the biasing member in the unexpanded state;
permitting the biasing member to move from the unexpanded state to an expanded state to move the first and second housing members in a direction away from each other;
contacting the contact surfaces of the first and second housing member against opposing body surfaces, the pivotal connection of the contact surfaces providing self-alignment of the contact surfaces with the opposing tissue surfaces.

20. The method of claim 19, wherein retaining the first and second housing member together includes engaging a release member with the first and second housing members, and releasing the first and second housing members includes disengaging the release member from at least one of the first and second housing members.

21. The method of claim 19, further comprising securing the first and second housing members together with at least one retaining member after the first and second housing members are moved in a direction away from each other.

22. The method of claim 19, wherein moving the first and second housing members in a direction toward each other includes inserting a portion of the first housing member into a portion of the second housing member.
